(19) United States
(12) Patent Application Publication
(10) Pub. No.: US 2021/0215630 A1
(43) Pub. Date: Jul. 15, 2021

(54) GAS SENSOR

(71) Applicant: NATIONAL CHIAO TUNG UNIVERSITY, Hsinchu City (TW)

(72) Inventors: Hsiao-Wen Zan, Hsinchu City (TW); Hong-Cheu Lin, Hsinchu City (TW); Hsin-Fei Meng, Hsinchu City (TW); Govindasamy Madhaiyan, Hsinchu City (TW)

(21) Appl. No.: 16/997,106

(22) Filed: Aug. 19, 2020

(30) Foreign Application Priority Data
Jan. 10, 2020 (TW) 109100833

(51) Int. Cl. G01N 27/30

(52) U.S. Cl. CPC G01N 27/30; G01N 33/0009

(57) ABSTRACT

A gas sensor includes a first electrode layer, a second electrode layer, and a gas sensing layer. The second electrode layer is spaced apart from the first electrode layer, has two electrode surfaces oppositely of each other, and is formed with a plurality of first through holes each extending through the two electrode surfaces. The gas sensing layer electrically interconnects the first electrode layer and the second electrode layer, and is made from a composition that includes a thiophene-based compound and a nitrogen-containing polar compound.

### CROSS-REFERENCE TO RELATED APPLICATION

This application claims priority of Taiwanese Patent Application No. 109100833, filed on Jan. 10, 2020.

### FIELD

The present disclosure relates to a sensor, and more particularly to a gas sensor.

### BACKGROUND

Taiwanese Invention Patent Publication No. 1615611 discloses a gas detector including an electrode unit that is adapted to be electrically connected to an electrical detector and a sensing unit. The electrode unit includes a first electrode layer and a second electrode layer that is spaced apart from the first electrode layer. The second electrode layer has two electrode surfaces oppositely of each other, and is formed with a plurality of through holes each extending through the electrode surfaces. The sensing unit includes a sensing layer for detecting a gas, which is connected to the first electrode layer and the second electrode layer. The sensing layer is made of a material, such as poly(9,9-dioctylfluorene-co-benzothiadiazole), poly[(4,8-bis[5-(2-ethylhexyl)thiophene-2-yl]benzo[1,2-b:4,5-b′]dithiophene)-2,6-diyl-alt-(4-(2-ethylhexanoyl)-thieno[3,4-b]thiophene))-2,6-diyl] (synonyms: poly[[4,8-bis[5-(2-ethylhexyl)-2-thienyl]benzo[1,2-b:4,5-b′]dithiophene-2,6-diyl][2-(2-ethyl-1-oxohexyl) thieno[3,4-b]thiophenediyl]]; PBDTTT-C-T), poly{4,8-bis(5-(2-ethylhexyl)thiophen-2-yl)benzo[1,2-b:4,5-b′]dithiophene-2,6-diyl-4-(2-ethylhexyloxycarbonyl)-3-fluoro-thieno[3,4-b]thiophene-2,6-diyl}, etc.

The gas detector disclosed in the aforesaid patent is capable of detecting amines (e.g., ammonia), aldehydes, ketones, nitric oxide, ethanol, nitrogen dioxide, carbon dioxide, ozone, a sulfide gas and other types of gases. However, the detection sensitivity and specificity of the gas detector is unsatisfactory. For example, when the gas detector is applied to detect nitric oxide in exhaled breath for facilitating diagnosis of respiratory diseases such as asthma, it would be susceptible to interference caused by ammonia that is usually present in the exhaled breath, resulting in inaccurate detection signal of nitric oxide.

### SUMMARY

Therefore, an object of the present disclosure is to provide a gas sensor that can alleviate at least one of the drawbacks of the prior art.

According to the present disclosure, the gas sensor includes a first electrode layer, a second electrode layer, and a gas sensing layer. The second electrode layer is spaced apart from the first electrode layer, has two electrode surfaces oppositely of each other, and is formed with a plurality of first through holes each extending through the two electrode surfaces. The gas sensing layer electrically interconnects the first electrode layer and the second electrode layer, and is made from a composition that includes a thiophene-based compound and a nitrogen-containing polar compound.

### BRIEF DESCRIPTION OF THE DRAWINGS

Other features and advantages of the present disclosure will become apparent in the following detailed description of the embodiments with reference to the accompanying drawings, of which:

FIG. 1 is a fragmentary schematic sectional view illustrating a first embodiment of a gas sensor according to the present disclosure;

FIG. 2 is a fragmentary schematic sectional view illustrating a second embodiment of the gas sensor according to the present disclosure;

FIG. 3 is a partial perspective view of FIG. 2;

FIG. 4 is a fragmentary schematic sectional view illustrating a third embodiment of the gas sensor according to the present disclosure; and

FIG. 5 is a fragmentary schematic sectional view illustrating a fourth embodiment of the gas sensor according to the present disclosure.

### DETAILED DESCRIPTION

Before the present disclosure is described in greater detail, it should be noted that where considered appropriate, reference numerals or terminal portions of reference numerals have been repeated among the figures to indicate corresponding or analogous elements, which may optionally have similar characteristics.

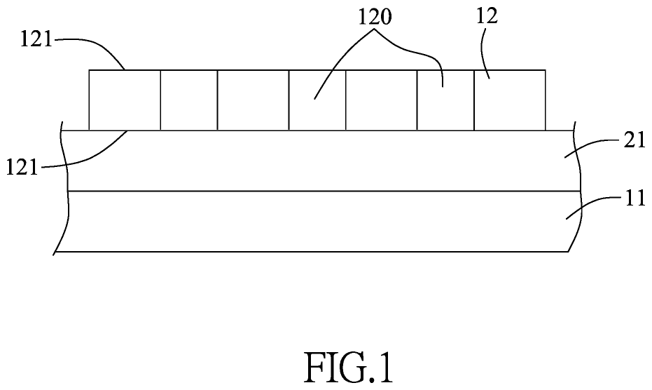

Referring to FIG. 1, a first embodiment of the gas sensor according to the present disclosure is configured to be electrically connected to an electrical detector (not shown in the figure). The electrical detector is capable of detecting electrical change when the gas sensor is in contact with a gas to be detected (such as nitric oxide). Examples of electrical change may include resistance change and current change. In an exemplary embodiment, the electrical change to be detected by the electrical detector is current change.

According to the present disclosure, the gas sensor includes a first electrode layer 11, a second electrode layer 12 that is spaced apart from the first electrode layer 11, and a gas sensing layer 21.

The first electrode layer 11 may have a length ranging from 1 mm to 10 mm, a width ranging from 1 mm to 10 mm, and a thickness ranging from 250 nm to 400 nm.

The second electrode layer 12 has two electrode surfaces 121 oppositely of each other, and is formed with a plurality of first through holes 120, each of which extends through the two electrode surfaces 121. The second electrode layer 12 may have a length ranging from 1 mm to 10 mm, a width ranging from 1 mm to 10 mm, and a thickness ranging from 350 nm to 1000 nm. Each of the first through holes 120 may independently have a diameter ranging from 50 nm to 200 nm.

The first and second electrode layers 11, 12 are independently made of a material that may include, a metal material, a metal compound material, and an organic conductive material, but is not limited thereto. Examples of the metal material may include, but are not limited to, aluminum, gold, silver, calcium, nickel, and chromium. Examples of the metal compound material may include, but are not limited to, indium tin oxide, zinc oxide, molybdenum oxide, and lithium fluoride. An example of the organic conductive material may include, but is not limited to, poly(3,4-ethylenedioxythiophene) polystyrene sulfonate (PEDOT:PSS). In the first embodiment, the first electrode layer 11 is made of indium tin oxide, and the second electrode layer 12 is made of aluminum. In a variation of the first embodiment, the second electrode layer 12 includes a plurality of interconnected nanowires.

The gas sensing layer 21, which is adapted for contacting gas, is disposed between and electrically interconnects the first electrode layer 11 and the second electrode layer 12. The gas sensing layer 21 may have a length ranging from 1 mm to 10 mm, a width ranging from 1 mm to 10 mm, and a thickness ranging from 200 nm to 400 nm. The gas sensing layer 21 is made from a composition that includes a thiophene-based compound and a nitrogen-containing polar compound.

Examples of the thiophene-based compound may include, but are not limited to, poly[[4,8-bis[5-(2-ethylhexyl)-2-thienyl]benzo[1,2-b:4,5-b′]dithiophene-2,6-diyl][2-(2-ethyl-1-oxohexyl) thieno[3,4-b]thiophenediyl]] (abbreviated as PBDTTT-C-T having the following formula (I)), poly{4,8-bis[(2-ethylhexyl)oxy]benzo[1,2-b:4,5-b′]dithiophene-2,6-diyl-alt3-fluoro-2-[(2-ethylhexyl)carbonyl]-thieno[3,4-b]thiophene-4,6-diyl} (abbreviated as PTB7 having the following formula (II)), poly(3-hexylthiophene-2,5-diyl) (abbreviated as P3HT having the following formula (III)), and combinations thereof.

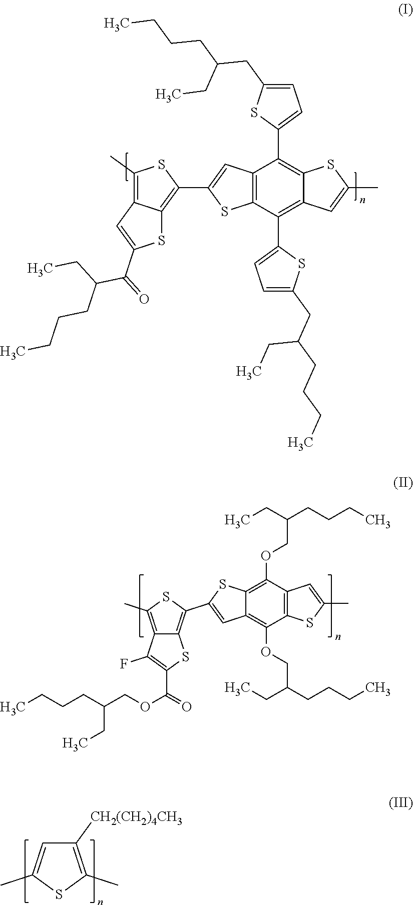

Examples of the nitrogen-containing polar compound may include, but are not limited to, spiropyran, 4-hydroxyazobenzene, N-ethyl-N-(2-hydroxyethyl)-4-(4-nitrophenylazo)aniline, and combinations thereof.

In certain embodiments, a weight ratio of the nitrogen-containing polar compound to the thiophene-based compound ranges from 0.5:10 to 3:10. In certain embodiments, the composition including the nitrogen-containing polar compound and the thiophene-based compound is subjected to ultraviolet light treatment to increase the polarity and to change the crystallinity along with the morphology of the thus made gas sensing layer 21, thereby improving the detection specificity and sensitivity to nitric oxide. In certain embodiments, the ultraviolet light treatment is performed at an irradiation wavelength that ranges from 200 nm to 400 nm, an irradiation energy that is greater than 10 mW/cm², and an irradiation time that is greater than 30 seconds. To be specific, the composition is first applied on the first and second electrode layers 11, 12 to form a coating film, which is then subjected to the ultraviolet light treatment, so as to obtain the gas sensing layer 21.

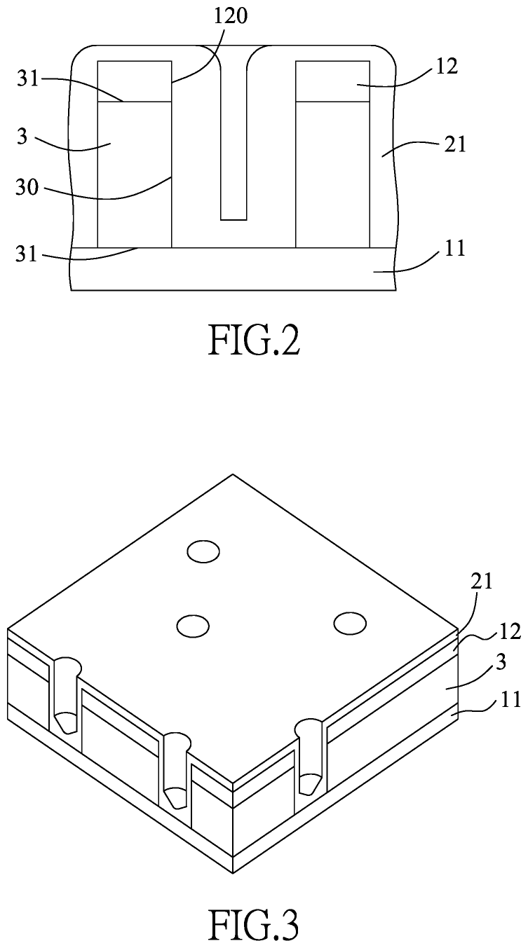

Referring to FIGS. 2 and 3, a second embodiment of the gas sensor according to the present disclosure is shown to be generally similar to the first embodiment, except for the following differences. To be specific, in the second embodiment, the gas sensor further includes a dielectric layer 3 that is disposed between the first electrode layer 11 and the second electrode layer 12. The dielectric layer 3 has two dielectric surfaces 31 oppositely of each other and is formed with a plurality of second through holes 30. Each of the second through holes 30 extends through the two dielectric surfaces 31 and is in spatial communication with a respective one of the first through holes 120. The gas sensing layer 21 is disposed on the second electrode layer 12, and extends into the first and second through holes 120, 30 to be electrically connected to the first electrode layer 11. That is, the first and second through holes 120, 30 are partially filled with the gas sensing layer 21.

The dielectric layer 3 may have a length ranging from 1 mm to 10 mm, a width ranging from 1 mm to 10 mm, and a thickness ranging from 200 nm to 400 nm. Each of the second through holes 30 may independently have a diameter ranging from 50 nm to 200 nm. The dielectric layer 3 is made of a material that may include, polyvinylphenol (abbreviated as PVP), polymethylmethacrylate (abbreviated as PMMA), a photoresist material, and polyvinyl alcohol (abbreviated as PVA), but is not limited thereto. An example of the photoresist material may include, but is not limited to, SU-8 negative photoresist (commercially available from M & R Nano Technology Co., Ltd., Taiwan). In this embodiment, the dielectric layer is made of polyvinylphenol (Manufacturer: Sigma Aldrich; Model No.: AL-436224) having a weight average molecular weight of 25000 Da.

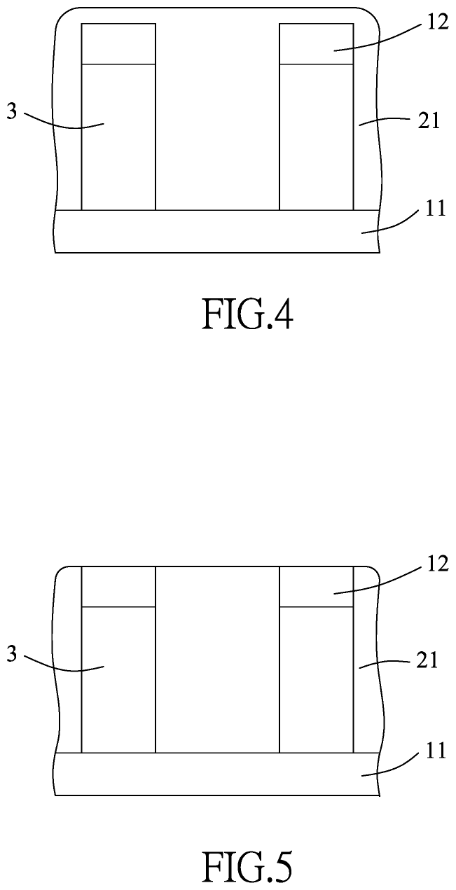

Referring to FIG. 4, a third embodiment of the gas sensor according to the present disclosure is shown to be generally similar to the second embodiment, except that, in the third embodiment, the gas sensing layer 21 is disposed on and covers the second electrode layer 12, and fills the first and second through holes 120, 30.

Referring to FIG. 5, a fourth embodiment of the gas sensor according to the present disclosure is shown to be generally similar to the third embodiment, except that, in the fourth embodiment, the gas sensing layer 21 is flushed with the second electrode layer 12.

The disclosure will be further described by way of the following examples. However, it should be understood that the following examples are intended solely for the purpose of illustration and should not be construed as limiting the disclosure in practice.

### EXAMPLES

### General Experimental Materials:

### 1. Thiophene-Based Compound

The thiophene-based compound used in the following examples includes poly[[4,8-bis[5-(2-ethylhexyl)-2-thienyl]benzo[1,2-b:4,5-bf]dithiophene-2,6-diyl][2-(2-ethyl-1-oxohexyl) thieno[3,4-b]thiophenediyl]] (Manufacturer: Solamer Materials, Inc.; weight average molecular weight: 20000 Da to 50000 Da), poly{4,8-bis[(2-ethylhexyl)oxy]benzo[1,2-b: 4,5-b′]dithiophene-2,6-diyl-alt3-fluoro-2-[(2-ethylhexyl)carbonyl]-thieno[3,4-b]thiophene-4,6-diyl}(Manufacturer: Solamer Materials, Inc.; weight average molecular weight: >23000 Da, poly(3-hexylthiophene-2,5-diyl) (Manufacturer: UniRegion Bio-Tech; Model No.: UR-P3H001; weight average molecular weight: 50000 Da to 70000 Da), which are respectively abbreviated as “PBDTTT-C-T”, “PTB7”, and “P3HT” in Table 1 below.

### 2. Nitrogen-Containing Polar Compound

The nitrogen-containing polar compound used in the following examples includes spiropyran (Manufacturer: Tokyo Chemical Industry Co., Ltd.; Model No.: T0423), 4-hydroxyazobenzene (kindly provided by Prof. Hong-Cheu Lin, Department of Materials Science and Engineering, National Chiao Tung University, Taiwan), and N-ethyl-N-(2-hydroxyethyl)-4-(4-nitrophenylazo)aniline (Manufacturer: Sigma Aldrich; Model No.: 344206).

### Examples 1 to 10 (EX1 to EX10) and Comparative Examples 1 to 6 (CE1 to CE6)

The gas sensors of EX1 to EX10 and CE1 to CE6 have the same structural configuration as shown in FIGS. 2 and 3 (i.e., the second embodiment as described above), and differ from one another only in terms of the composition and procedures for making the gas sensing layer 21 thereof (see Table 1). To be specific, each of the gas sensing layers 21 in EX1 to EX10 was made from the composition including the specified thiophene-based compound and nitrogen-containing polar compound in a weight ratio of 10:1, while each of the gas sensing layers 21 in CE1 to CE10 was made from the composition merely including the specified thiophene-based compound. In addition, each of the compositions used in EX1, EX3, EX5, EX7, EX9, CE1, CE3 and CE5 was further subjected to an ultraviolet light treatment that was performed at an irradiation wavelength of 365 nm, an irradiation energy of 40 mW/cm², and an irradiation time of 300 seconds.

TABLE 1

| Gas | Composition | | |
|---|---|---|---|
| sensing | Thiophene-based | Nitrogen-containing | UV light |
| layer | compound | polar compound | treatment |
| EX1 | PBDTTT-C-T | Spiropyran | + |
| EX2 | PBDTTT-C-T | Spiropyran | − − |
| EX3 | PBDTTT-C-T | 4-hydroxyazobenzene | + |
| EX4 | PBDTTT-C-T | 4-hydroxyazobenzene | − − |
| EX5 | PBDTTT-C-T | N-ethyl-N-(2- | + |
| | | hydroxyethyl)-4-(4- | |
| | | nitrophenylazo)aniline | |
| EX6 | PBDTTT-C-T | N-ethyl-N-(2- | − − |
| | | hydroxyethyl)-4-(4- | |
| | | nitrophenylazo)aniline | |
| EX7 | PTB7 | Spiropyran | + |
| EX8 | PTB7 | Spiropyran | − − |
| EX9 | P3HT | 4-hydroxyazobenzene | + |
| EX10 | P3HT | 4-hydroxyazobenzene | − − |
| CE1 | PBDTTT-C-T | — | + |
| CE2 | PBDTTT-C-T | — | − − |
| CE3 | PTB7 | — | + |
| CE4 | PTB7 | — | − − |
| CE5 | P3HT | — | + |
| CE6 | P3HT | — | − − |

″—″: not added; ″− −″: not performed

In testing, each of the gas sensors of EX1 to EX10 and CE1 to CE10 was placed in a chamber filled with air, and the first electrode layer 11 and the second electrode layer 12 were electrically connected to an external electrical device (Manufacturer: Keithley Instruments; Model No.: U2722A) that includes a voltage supply for providing an applied voltage and a current detector for detecting current change. The applied voltage of each of the gas sensors of EX1 to EX10 and CE1 to CE10 was shown in Table 2. Next, a gas to be tested (i.e., ammonia (NH₃) or nitric oxide (NO) in a specific concentration (i.e., 100, 300, 500 and 1000 ppb) was introduced into the chamber to contact with the gas sensors of the respective one of EX1 to EX10 and CE1 to CE10 for 60 seconds, and the current was traced using the current detector. The current change percentage for the gas sensors of each of EX1 to EX10 and CE1 to CE10 before and after introduction of a respective one of NH₃ and NO in a specified concentration was calculated using the following formula:

A=[(B−C)/C]×100%

where A=current change percentage
B=current value at the end of the predetermined contact time period
C=current value prior to introduction of NH₃ or NO.

The thus calculated current change percentage and the ratio of the current change percentage of NO to that of NH₃ for the gas sensors of each of EX1 to EX10 and CE1 to CE10 are shown in Table 2 below.

TABLE 2

| | | | | | | | | | | Ratio of | |
|---|---|---|---|---|---|---|---|---|---|---|---|
| | | | | | | | | | | current | |
| | | | | | | | | | | change | |
| | | Current change percentage (%) | | | | | | | | percentage | |
| | | Concentration | | | | Concentration | | | | of NO | |
| | | of NH₃ | | | | of NO | | | | to NH₃ | |
| | | introduced | | | | introduced | | | | Without | |
| | Applied | into the | | | | into the | | | | UV | With UV |
| | voltage | chamber (ppb) | | | | chamber (ppb) | | | | light | light |
| | (V) | 100 | 300 | 500 | 1000 | 100 | 300 | 500 | 1000 | treatment | treatment |
| EX1 | 18 | n.d. | n.d. | 0.1 | 4.5 | 7.9 | 27.0 | 39.2 | 76.0 | — | 16.89 |
| EX2 | 18 | n.d. | n.d. | 3.7 | 8.5 | n.d. | n.d. | 12.0 | 24.5 | 2.88 | — |
| EX3 | 8 | n.d. | n.d. | n.d. | 16.3 | 8.7 | 29.2 | 40.2 | 75.0 | — | 4.60 |
| EX4 | 8 | n.d. | n.d. | n.d. | 12.1 | 12.3 | 39.8 | 57.5 | 91.9 | 7.60 | — |
| EX5 | 10 | n.d. | n.d. | n.d. | 5.4 | 1.6 | 5.1 | 7.7 | 13.2 | — | 2.44 |
| EX6 | 10 | n.d. | n.d. | n.d. | 5.5 | 1.5 | 5.3 | 8.9 | 18.7 | 3.40 | — |
| CE1 | 5 | n.d. | n.d. | n.d. | 38.0 | 31.4 | 74.5 | 142 | 233 | — | 6.13 |
| CE2 | 5 | n.d. | n.d. | n.d. | 40.4 | 10.2 | 25.9 | 49.3 | 93.0 | 2.30 | — |
| EX7 | 6 | n.d. | 10.8 | 14.1 | 21.8 | 20.0 | 57.4 | 82.3 | 144 | — | 6.61 |
| EX8 | 6 | n.d. | n.d. | 7.2 | 21.4 | 11.3 | 56.1 | 73.1 | 136 | 6.36 | — |
| CE3 | 6 | n.d. | n.d. | 9.8 | 22.9 | 4.9 | 14.3 | 25.3 | 29.3 | — | 1.28 |
| CE4 | 6 | n.d. | n.d. | 7.6 | 22.4 | 7.8 | 22.8 | 38.2 | 59.0 | 2.63 | — |
| EX9 | 2~3 | n.d. | n.d. | 1.8 | 4.7 | 1.2 | 6.6 | 13.3 | 35.1 | — | 7.47 |
| EX10 | 2~3 | n.d. | n.d. | 0.6 | 4.4 | 3.0 | 11.5 | 23.5 | 85.5 | 19.43 | — |
| CE5 | 2 | n.d. | n.d. | n.d. | 13.4 | 5.8 | 17.9 | 31.2 | 51.1 | — | 3.81 |
| CE6 | 2 | n.d. | n.d. | n.d. | 9.8 | 2.4 | 9.3 | 25.9 | 39.7 | 4.05 | — |

“n.d.”: not detected; “—”: not performed

As shown in Table 2, the gas sensors of EX2, EX4 and EX6, each of which has a gas sensing layer made from a composition that includes a thiophene-based compound (i.e., PBDTTT-C-T) and a nitrogen-containing polar compound without being subjected to ultraviolet (UV) light treatment, have significantly higher ratios of current change percentage of NO to that of NH₃ as compared to that of the gas sensor of CE2 having a gas sensing layer made from only a thiophene-based compound. Similarly, as compared to CE4 and CE6, the gas sensors of EX8 and E10, each of which has a gas sensing layer made from the thiophene-based compound and the nitrogen-containing polar compound without being subjected to the UV light treatment, have significantly higher ratios of the current change percentage of NO to that of NH₃. These results indicate that inclusion of the nitrogen-containing polar compound in the composition for making the gas sensing layer improves the specificity of the gas sensors for detecting NO and reduces interference caused by NH₃.

In addition, the gas sensors of EX1, EX3 and EX5, each of which has a gas sensing layer made from the composition that is similar to those in EX2, EX4 and EX6 and that is subjected to UV light treatment, show significantly higher ratios of the current change percentage of NO to that of NH₃ as compared to the gas sensor of CE2 having a gas sensing layer made from the composition without being subjected to the UV light treatment. Similarly, as compared to CE3 to CE6, the gas sensors of EX7 and EX9, each of which has a gas sensing layer made from the composition that includes the thiophene-based compound and the nitrogen-containing polar compound and that is subjected to the UV light treatment, have significantly higher ratios of the current change percentage of NO to that of NH₃. These results indicate that inclusion of the nitrogen-containing polar compound in the composition and then subjecting the composition to the UV light treatment for making the gas sensing layer 21 may further improve the specificity of the gas sensors for detecting NO.

In summary, through the gas sensing layer 21 that is made from the thiophene-based compound and the nitrogen-containing polar compound, the gas sensor of this disclosure has increased specificity and sensitivity for detecting a gas of interest (such as NO).

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the present disclosure has been described in connection with what is considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A gas sensor, comprising:
a first electrode layer,
a second electrode layer being spaced apart from said first electrode layer, having two electrode surfaces oppositely of each other, and being formed with a plurality of first through holes each extending through said two electrode surfaces; and
a gas sensing layer electrically interconnecting said first electrode layer and said second electrode layer, and being made from a composition that includes a thiophene-based compound and a nitrogen-containing polar compound.

2. The gas sensor as claimed in claim 1, wherein said nitrogen-containing polar compound is selected from the group consisting of spiropyran, 4-hydroxyazobenzene, N-ethyl-N-(2-hydroxyethyl)-4-(4-nitrophenylazo)aniline, and combinations thereof.

3. The gas sensor as claimed in claim 1, wherein said thiophene-based compound is selected from the group consisting of poly[[4,8-bis[5-(2-ethylhexyl)-2-thienyl]benzo[1,2-b:4,5-b′]dithiophene-2,6-diyl][2-(2-ethyl-1-oxohexyl) thieno[3,4-b]thiophenediyl]], poly{4,8-bis[(2-ethylhexyl)oxy]benzo[1,2-b:4,5-b′]dithiophene-2,6-diyl-alt3-fluoro-2-[(2-ethylhexyl)carbonyl]-thieno[3,4-b]thiophene-4,6-diyl}, poly(3-hexylthiophene-2,5-diyl), and combinations thereof.

4. The gas sensor as claimed in claim 1, wherein said composition is subjected to ultraviolet light treatment to make said gas sensing layer.

5. The gas sensor as claimed in claim 4, wherein said nitrogen-containing polar compound is selected from the group consisting of spiropyran, 4-hydroxyazobenzene, N-ethyl-N-(2-hydroxyethyl)-4-(4-nitrophenylazo)aniline, and combinations thereof.

6. The gas sensor as claimed in claim 5, wherein said thiophene-based compound is selected from the group consisting of poly[[4,8-bis[5-(2-ethylhexyl)-2-thienyl]benzo[1,2-b:4,5-b′]dithiophene-2,6-diyl][2-(2-ethyl-1-oxohexyl) thieno[3,4-b]thiophenediyl]], poly{4,8-bis[(2-ethylhexyl)oxy]benzo[1,2-b:4,5-b′]dithiophene-2,6-diyl-alt3-fluoro-2-[(2-ethylhexyl)carbonyl]-thieno[3,4-b]thiophene-4,6-diyl}, poly(3-hexylthiophene-2,5-diyl), and combinations thereof.

7. The gas sensor as claimed in claim 1, wherein said gas sensing layer is disposed between said first electrode layer and said second electrode layer.

8. The gas sensor as claimed in claim 1, further comprising a dielectric layer that is disposed between said first electrode layer and said second electrode layer, that has two dielectric surfaces oppositely of each other and that is formed with a plurality of second through holes, each of said second through holes extending through said two dielectric surfaces and being in spatial communication with a respective one of said first through holes.

9. The gas sensor as claimed in claim 8, wherein said gas sensing layer extends into said first and second through holes to be electrically connected to said first electrode layer.

10. The gas sensor as claimed in claim 9, wherein said gas sensing layer covers said second electrode layer.

11. The gas sensor as claimed in claim 9, wherein said gas sensing layer fills said first and second through holes, and is flushed with said second electrode layer.
